(19) United States
(12) Patent Application Publication
(10) Pub. No.: US 2019/0218487 A1
(43) Pub. Date: Jul. 18, 2019

(54) METHOD FOR REFINING RECTIFIED ALCOHOL AND APPARATUS FOR IMPLEMENTING SAME

(71) Applicant: Stanislav Veniaminovich SAVINSKYI, Kiev (UA)

(72) Inventor: Stanislav Veniaminovich SAVINSKYI, Kiev (UA)

(21) Appl. No.: 16/324,676

(22) PCT Filed: Feb. 27, 2017
(86) PCT No.: PCT/UA2017/000017

§ 371 Date: Feb. 11, 2019

(30) Foreign Application Priority Data
Aug. 10, 2016 (UA) A 2016 08698

(51) Int. Cl. C12G 3/04; C12H 6/02; B01D 3/26; B01F 3/20

(52) U.S. Cl. CPC C12G 3/04; C12H 6/02; B01D 3/26; C07C 31/08; B01F 3/2014; B01F 2215/007; B01F 3/2078

(57) ABSTRACT

The present invention relates to the field of food industry and to pharmacology and medicine, and it is intended for refining water-alcohol mixtures by the way of de-rectifying and homogenizing those mixtures, and also it will find a wide application in manufacturing and processing beverages or liquid medicaments based on rectified spirit. The objective of the invention is to create such a method for refining rectified alcohol, which would ensure maximum improvement of a beverage by small changes in its structure while maintaining the original taste of the initial product, as well as creating a device for refining rectified alcohol that would ensure the high efficiency of the processes for de-rectifying and homogenizing of the beverage evenly throughout the rectifying column with minimal production costs.

### CROSS REFERENCE TO RELATED APPLICATION

This application is a national stage entry of PCT/UA2017/000017 filed Feb. 27, 2017, under the International Convention claiming priority over Ukraine Patent Application No. a 2016 08698 filed Oct. 10, 2016.

### TECHNICAL FIELD

The present invention relates to the field of food industry and to pharmacology and medicine, and it is intended for refining water-alcohol mixtures by the way of de-rectifying and homogenizing those mixtures, and also it will find a wide application in manufacturing and processing beverages or liquid medicaments based on rectified alcohol.

### BACKGROUND

The term of ‘rectified alcohol’ hereinafter means a water-alcohol solution prepared with the use of rectified ethanol, which may contain various ingredients, and is intended for consumption as an alcoholic beverage or medicines. The rectified alcohol includes vodka, whiskey, blended with neutral (190 proof) alcohol, gin, liqueurs, alcoholates and extracts, as well as any other alcoholic beverages and drinking medicines containing rectified spirit.

According to the Merriam-Webster dictionary, the term of ‘refining,’ means a process for removing unwanted substances from something, or improving something by making small changes therein. Therefore, the term of ‘refining’ hereinafter means the nano-process for improving the rectified alcohol in the course of providing its processing at heat and mass transfer apparatuses for de-rectifying ethanol, in order to reduce harmful effects of the rectified alcohol on the human body. In the context of this document, the concept of ‘refining’ also includes the simultaneous process of homogenizing the water-alcohol mixture at the column of the apparatus in order to improve the organoleptic properties of the drink.

The term of ‘de-rectifying’ hereinafter means the process of combining spirit with water, that is a process being reverse to a rectifying one, which occurs in a rectifying column in the course of processing the water-alcohol mixtures in the reverse direction downward or horizontally in the direction perpendicular to the temperature gradient in the column. The intense oscillations of the ethanol and water molecules caused by the high temperature of the de-rectifying process, and the numerous phase transitions of the mixture in the rectifying column ensure occurring the process of effective decomposition of the rectified ethanol clusters, which are harmful for humans, into individual molecules, as well as the process of formation of ethanol crystalline hydrates, which are typical for the natural ethanol fermentation process.

The term of ‘homogenizing’ hereinafter means the process of mixing and uniformly distributing the particles of the rectified alcohol ingredients, including the ethanol crystalline hydrates, throughout the phlegma volume, that process being occurred as a result of intense Brownian motion caused by high temperature of heat and mass transfer processes, and numerous phase transitions of the drink water and spirit mixture in the rectifying column.

The term of ‘packing’ hereinafter means a special set of contact elements, which fill the inner space of the column to intensify the process of heat and mass transfer by increasing the surface of the phase interactions.

It is well known that rectified ethanol is not only mixed with water, but it is combined with the same by the way of contraction, which is accompanied by the formation of the ethanol crystallohydrates. It is known that the process of simple mechanical mixing of rectified spirit with water makes it difficult to obtain the ethanol uniformly solvated with a hydrated shell. It has also been proved that ethanol hydrates are more easily formed in the gas phase than in the liquid one, and that such a process takes place in the course of condensing of water vapor and ethanol in heat and mass transfer apparatuses.

There is a process and apparatus for refining a strong alcoholic beverage by repeated distillation, proposed by Joseph Avalov (Awaloff, German Patent No. 599,498 issued Jul. 3, 1934). The process proposes a stream of vodka, usually of about 40% strength, to be fed from a reservoir into the middle part point of a rectifying column at different distances from the column upper end, depending on the content of fusel-oils in the initial product. At the upper end of the rectifying column, there is a water cooler providing a constant level for a condensate of a water-ethanol solution. Into the lower part of the column, there is fed water vapor, which as rising up through the column, feeds it with water and heat and, therefore, creates a temperature gradient along the rectifying column.

That is, in the apparatus, the water-ethanol product moves vertically along the temperature gradient in the upstream direction generally accepted for the standard rectification or distillation apparatuses.

The secondary process of rectifying the water-ethanol product on the trays of the rectifying column for the apparatus leads to condensing and concentrating ethanol vapor and separating the fusel-oils, which flow together with the phlegma into the reservoir under the rectifying column. The rectification rate is controlled by a contact thermometer designed for controlling the heat supply, while the input flow rate feeding the reservoir is carefully controlled according to the distillation rate, which fact allows setting and controlling the concentration of spirit at the outlet.

However, the disadvantage of this process is the fact that distillation, as well as rectification, is the process of separation of liquid mixtures consisting of components with different boiling points. It is due to the predominance of the processes of separation of ethanol from water over the processes of their condensation, there is possible the very fact of the separation of ethanol from distillery stillages during the process of distillation. Therefore, even theoretically, the secondary distillation cannot be effectively used to combine spirit with water, and the rectified alcohol obtained with the help of such a technological procedure may contain clusters of non-hydrated ethanol molecules that negatively affect the human body. The additional disadvantage of this device is also a complexity of its design and the fact of its unsuitability for preparing of a sufficiently homogeneous water-ethanol solution. Moreover, the repeated distillation leads to the removal of additional ingredients off the drink, primarily the salts of the water component, which results in the loss of the original taste of the initial product.

The closest technical solution to the present method of refining the rectified alcohol is the method described in the document [Patent of Ukraine No. 104536, IPC: CCC12G 3/04, B01F 13/02, BO1D 3/26. Publ.10.01.01.2014]. In this method, the water-alcohol mixture is heated and, in the form of vapor, supplied in the same direction with the phlegma along the packing from top to bottom and the condensate of the water-alcohol mixture, in the form of the phlegma, is sent from the warmer portion of the apparatus to the cooler one of the same. The finished sorting is collected in the form of the phlegma at the bottom outlet of the column. In this case, the cooling agent is fed to the apparatus packing from the bottom up to the top, in the direction toward the flow of heat and phlegma creating a temperature gradient along the rectifying column.

That is, the water-ethanol product in this apparatus is fed not only in the downward direction opposite to that generally accepted for the standard distillation or rectification units, but also vertically and along the temperature gradient.

The disadvantage of this method is that, with the opposite flows of heat and cooling agent along the column, a temperature gradient is created with an optimum for heat-mass transfer processes only in one zone of the column, while the rest of the column space operates outside the optimum temperatures, which fact leads to non-sufficient effectiveness of the process for refining rectified alcohol in such a way.

The closest technical solution to the apparatus for refining rectified alcohol according to the described method is a heat and mass transfer apparatus with a vertical rectifying column for producing vodka sorting [Patent of Ukraine No. 104536, IPC: CCC12G 3/04, B01F 13/02, B01D 3/26. Publ.10.01.01.2014].

The apparatus for the implementation of the described method consists of a rectifying column with packing, the rectifying column case is equipped with a cooling jacket and branch pipes for supplying the initial product and cooling agent and for discharging the finished product in the form of vodka sorting phlegma and heated water, while in the upper portion of the rectifying column, there are arranged the feeding branch pipes for the initial product and the branch pipe for discharging the heated cooling agent, and at the bottom portion of the rectifying column, there is a pipe for feeding the cooling agent and a branch pipe for discharging the finished product in the form of the vodka sorting phlegma. In addition, in the lower end portion, the apparatus is equipped with a refrigerator-condenser connected to the outlet brunch pipe for discharging the vodka sorting in the form of phlegma. In this case, the brunch pipe for discharging the heated cooling agent is connected with the branch pipe for feeding the initial product through a heat exchanger.

The disadvantage of this apparatus is that such a design provides for the formation of a vertical temperature gradient along the height of the column, and it does not allow maintaining the identical and stable conditions for heat and mass transfer processes along the entire length of the rectifying column, which condition is one of the main requirements to provide the high efficiency of the processes for de-rectifying and homogenizing of the rectified alcohol.

### SUMMARY

The objective of the invention is to create such a method for refining a rectified alcohol, which would ensure maximum improvement of its quality to reduce the harmful effects of the rectified alcohol on the human body with simultaneously maintaining the original taste of the initial product, as well as creating a device for refining the rectified alcohol that would ensure the high efficiency of the processes for de-rectifying and homogenizing the same evenly throughout the phlegmator column with minimal production costs.

The technical problem is solved by the inventive method for refining a rectified alcohol by passing the same through a rectifying column and separating its the condensate in the form of phlegma, wherein the rectified alcohol at a specified speed is fed into the head portion of the column, within which a given temperature gradient is maintained, and the finished product is collected at the outlet of the rectifying column in the form of phlegma, while the difference of the claimed invention is that the temperature gradient is created across the width of the rectifying column, and the rectified is directed horizontally along the length of the rectifying column and perpendicularly with respect to the temperature gradient.

The rectified alcohol is passed at a constant speed along the horizontally arranged rectifying column, which is heated along the entire length on one side of the rectifying column up to the state of boiling the liquid in the column and which is simultaneously cooled on the opposite side of the rectifying column until the vapor in the rectifying column condenses into the phlegma. At the same time, there is provided continuous movement of the two phases from the head portion of the rectifying column to the outlet portion thereof.

That is, in comparison with the upward direction for the movement of the water-alcohol mixture and heat towards the phlegma, which is generally accepted in the art and also same as downward direction of the water-alcohol mixture and heat movement along the phlegma in the prototype, the inventive technical problem is solved by changing for the horizontal direction of the water alcohol mixture movement across the temperature gradient. In all the opposed technical solutions, the movement of the water-alcohol mixture in the rectifying column occurs along the temperature gradient. No technical solution in the art uses the horizontal arrangement for a rectifying column. Such a difference feature allows achieving the maximum level of de-rectifying and homogenizing of the rectified alcohol with reducing the harmful effects of the rectified alcohol on the hukman body, as well as it provides, as well as improving the organoleptic properties of the drink while maintaining the original taste of the initial product.

The claimed inventive problem is also solved by the inventive apparatus, which consists of a horizontal column with end openings for supplying the initial product and discharging the finished product in the form of phlegm, as well as heating and cooling elements, while the difference of the claimed device consists in that along the entire length of the column, at is opposite sides, there are arranged heating and cooling elements, and the heating element is arranged on the bottom or side portion, and the cooling element is arranged on the top or side portion of the column, respectively.

This difference feature allows creating a temperature gradient across the rectifying column width in the direction perpendicular to the direction of movement of the water-alcohol mixture of the rectified alcohol along the column, and maintaining the identical temperature conditions in all the zones along the length of the rectifying column, which fact provides for equally high efficiency of the processes for de-rectifying and homogenizing the rectified alcohol along the entire length of the rectifying column.

The second additional difference feature of the claimed apparatus is that the rectifying column is designed in the form of a horizontal hollow plate, on the lower side of which, the heating element is arranged throughout the area thereof, and on the upper side of the plate, the cooling element is arranged throughout the area thereof.

This difference feature allows increasing the efficiency of the apparatus and it is expedient for industrial use of the method, for example, in the production of alcoholic beverages.

The third additional difference feature consists in that the rectifying column is made in the form of a tube twisted into a flat horizontal spiral shape, under which the heating element is arranged and above which the cooling element is arranged.

This difference feature allows increasing the length of the rectifying column and enhancing the efficiency of the apparatus while reducing its overall dimensions. This fact makes it possible to use the method for deeper refining of the product, for example, in the event of producing or processing of drinking medicines containing rectified spirit.

The fourth additional difference feature consist of that the rectifying column is made in the form of a tube spirally twisted around the vertical heating element, and the cooling element is made in the form of a fan.

This difference feature increases the efficiency of the apparatus by using the initial rectified alcohol as a cooling agent, which feature also provides preheating of the initial solution before it turns into steam. Moreover, this difference feature creates the possibility for producing compact apparatus with air-cooling, which fact is convenient for the use in domestic conditions.

Thus, the apparatus for refining rectified alcohol consists of a reservoir for storing a rectified alcohol, horizontally arranged rectifying column, and a pump for controlled supply of the solution to the head portion of the column. In the case of supplying the rectified alcohol to the column by gravity, that is under the force of gravity, instead of a pump, the apparatus is equipped with a control valve. The opposite sides of the rectifying column, along the entire length thereof, are equipped with heating and cooling elements. To increase the intensity of formation of the phlegma, the rectifying column can be equipped with a packing, which increases the surface areas for boiling the solution and condensing the phlegma. To provide final cooling of the finished product transferred into the phlegma, the apparatus is equipped with a special cooling element associated with the vessel for collecting the finished product.

The proposed method and the apparatus for implementing thereof provide refining rectified alcohol by reverse rectification process of multistage combining of ethanol with water in the apparatus, which ensures the movement of the liquid and gas phases along the column: firstly, in the horizontal direction; secondly, in the direction perpendicular to the temperature gradient; thirdly, in the direction perpendicular to the ascending vapors of the boiling water-alcohol mixture; in the fourth, in the direction perpendicular to the downward flow of the phlegma; in the fifth, on the same temperature conditions along the entire length of the rectifying column.

The regulated supply of the rectified alcohol to the head portion of the rectifying column is executed by gravity or by a pump. The heating element of the rectifying column provides the uniform process for boiling the rectified alcohol along the entire length of the rectifying column, and the cooling element provides the uniform process of condensing the vapors and forming the phlegma along the entire length of the rectifying column. The phlegma flows down over the side walls and along the packing of the rectifying column, being constantly enriched with oncoming vapor of alcohol and water, and the reflux number of the apparatus tends to infinity. At the same time, under the action of the pump, the two phases, both liquid and gaseous ones, simultaneously move from the head portion of the rectifying column to the outlet of the same. Residual vapors of the rectified alcohol are condensed on the cooling element of the apparatus and mixed with the phlegma. The finished refined alcohol in the form of the cooled phlegma is collected at the outlet of the column.

The apparatus productivity depends on its geometrical parameters and is regulated by the feed flow rate of the initial solution provided by the pump or the control valve and by the temperature values of the heating and cooling elements.

The inventive method of refining rectified alcohol and the apparatus for its implementation are new, because such technical solutions were not known to the author and the applicant on the date of filing the application from the available sources of information.

The inventive method for refining rectified alcohol and the apparatus for its implementation have an inventive step, because before the filing date of this application, with minimum investments and achieving maximum de-rectification and homogeneity of the rectified alcohol reducing the harmful effects of the rectified spirit on the human body and improving the organoleptic qualities of the drink while maintaining the original taste of the initial product, none of the specialists in the art had proposed a similar solution.

The inventive method of refining rectified alcohol and the apparatus for its implementation are industrially suitable, since a skilled specialist has no doubts about the possibility of their introduction into production and life. The invention in any form of embodiments of the inventive concept can be industrially realized using conventional means in mechanical engineering.

### BRIEF DESCRIPTION OF DRAWINGS

The inventive method of refining rectified alcohol and the apparatus for its implementation are explained by the drawings, which are schematically depicted in:

FIG. 1. shows a schematic diagram of the inventive apparatus;

FIG. 2. shows a cross-section view of a horizontal plate-type rectifying column with bottom heating and top cooling;

FIG. 3. shows a cross-section of a horizontal cylindrical rectifying column with side heating and side cooling;

FIG. 4. shows an apparatus for refining alcoholic beverages according to the claimed method;

FIG. 5. shows an embodiment of the apparatus; and.

FIG. 6 shows the effects of processing rectified alcohol after the prototype and the claimed method on precipitation of pg red blood cells.

In this case, the symbols indicated in the figures show as follows:

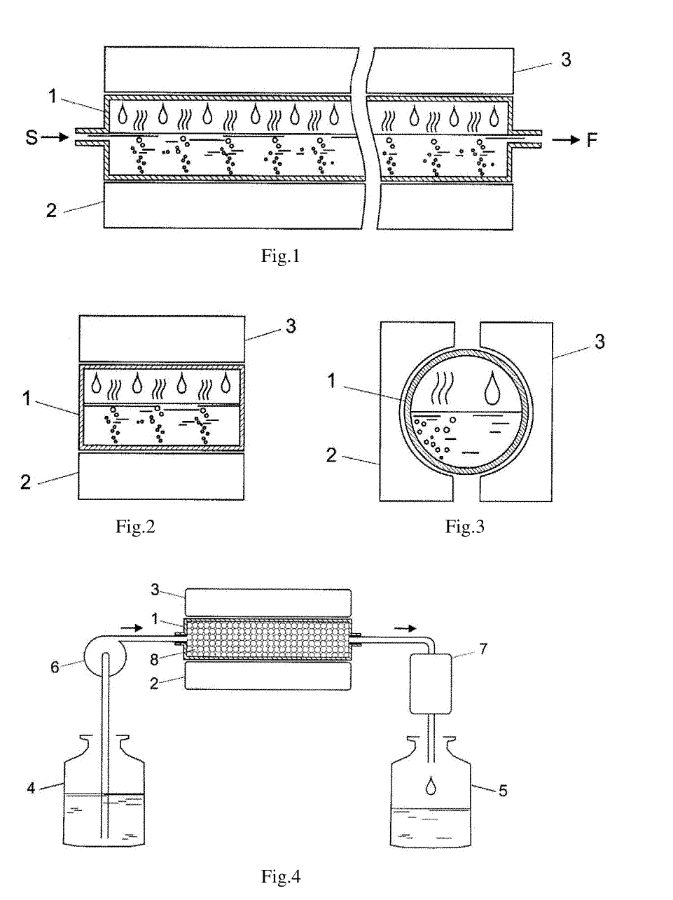

FIG. 1. Schematic diagram of the inventive apparatus:
1. Rectifying column.
2. Heating element.
3. Cooling element.
(8) Output rectified alcohol output solution.
(P) Finished product (refined alcohol phlegma).

FIG. 2. The cross-section of the horizontal plate-type column with bottom heating and top cooling.
1. Rectifying column.
2. Heating element.
3. Cooling element.

FIG. 3. Cross-section of the horizontal cylindrical column with lateral heating and cooling.
1. Rectifying column.
2. Heating element.
3. Cooling element.

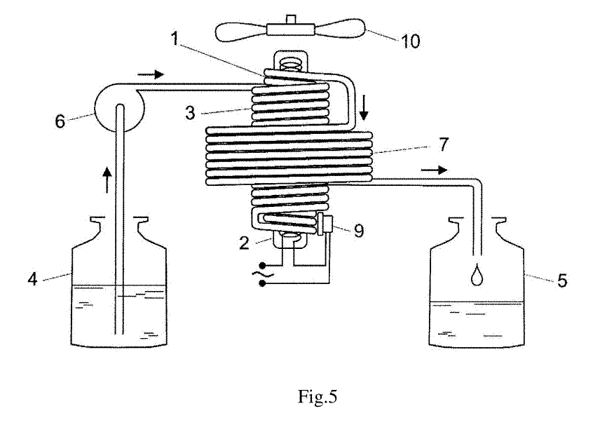

FIG. 4. Apparatus for refining the alcoholic beverages according to the claimed method:
1. Rectifying column.
2. Heating element of the rectifying column.
3. Cooling element of the rectifying column.
4. Reservoir for the initial product.
5. Reservoir for the finished product.
6. Pump for controlled feed of the initial product.
7. Cooling element for the finished product.
8. Rectifying column packing.

FIG. 5. The apparatus embodiment
1. Rectifying column.
2. Heating element of rectifying column.
3. Cooling element for the rectifying column.
4. Reservoir for the initial product.
5. Reservoir for the finished product.
6. Pump for controlled feed of the initial product.
7. Cooling element for the finished product.
8. Thermorelay for heating element.
9. Cooling fan.

The operation of the claimed device and the process for its application.

The essence of the proposed method for refining rectified alcohol consists in the fact that in order to obtain a high-quality water-alcohol mixture of a drink, there is used a process inverse to the process of rectification of ethyl alcohol.

It is known that the undoubted condition for effective separating alcohol from water in a rectifying column is a provision of a process wherein a water-alcohol product is fed vertically and along the temperature gradient, whereas, in the inventive method, to combine the alcohol with water, the water-alcohol mixture is directed along the horizontal column in the direction perpendicular to the temperature gradient.

At the same time, in the course of the phase transitions of the solution ingredients, at the level of molecules, there is achieved a destruction of ethanol clusters and a formation of ethanol crystalline hydrates, as well as it is provided a uniform distribution of the mixture particles over the finished product volume.

The present method and the device for its implementation propose to pass the rectified alcohol through a horizontal rectifying column, the opposite sides of which are heated and, accordingly, cooled along the entire length of the rectifying column to create a transverse temperature gradient over the entire length of the column. This inventive feature provides for the continuous movement of the liquid and gas phases of the drink in the horizontal direction and perpendicularly to the direction of the upward movement of the solution vapors and the downward movement of the phlegma, with the finished product being collected at the outlet of the column in the form of phlegma. Due to the increased temperature in the rectifying column, the kinetic energy of the molecular oscillations increases, which fact contributes to the disintegration of the agglomerates of the water-alcohol mixture ingredients. In the gas phase, there is occurred a constant separation of the rectified ethanol clusters into individual molecules, which are solvated by the water molecules in the course of condensing the vapors into the phlegma. This results in effectively combining the alcohol with water at the molecular level. Such a method and a design of the apparatus for its implementation provide a constant temperature of the liquid and gas phases along the entire length of the column, which fact increases the efficiency of using the entire volume of the column. A large number of phase transitions of a water-ethanol solution provide for the effective formation of the ethanol crystalline hydrates, which are characteristic for the ethanol of the natural fermentation process, and their uniform distribution over the volume of the finished product.

The essence of the apparatus for refining the rectified alcohol with the use of the method being claimed is as follows:

The apparatus for refining rectified alcohol (see FIGS. 1, 2) consists of horizontal rectifying column 1, at the bottom potion of which and along the entire side thereof, there is arranged a heating element, for example, a steam, oil, electric, or induction, and so on, element 2 of above said rectifying column 1. At the top portion and along the entire side of the column, there is arranged cooling element 3 of column 1, for example, with water, oil or another cooling agent. Heating element 2 and cooling element 3 of the rectifying column 1 can also be arranged on the sides of column 1 (see FIG. 3) in the horizontal plane. At the inlet of rectifying column 1, there is located reservoir 4 for storing the water-alcohol mixture, and at the outlet of rectifying column 1, there is placed reservoir 5 for the finished product. At the same time, the cavity of reservoir 4 is connected to the cavity of column 1 owing to the availability of pump 6, which is designed to control feeding of the initial solution to the head of the rectifying column, and at the outlet of the column, to provide the procedure of conclusively cooling the finished product transferred into the phlegma, the cavity of the rectifying column may be associated with the cavity of reservoir 5 for the finished product through cooling element 7 for the finished product. To increase the intensity of the phlegma formation, rectifying column 1 can be provided with packing 8 (see FIG. 4), which increases the surface area for boiling the solution and condensing the phlegma. In addition, (see FIG. 5) a thermo regulator or thermorelay 9 can be installed on the column to automatically control the heating and cooling temperatures, and fan 10 can be arranged above cooling element 7 to cool the finished product. In this case, rectifying column 1 can be made in the form of a horizontally arranged hollow plate with inlet and outlet branch pipes, as shown in FIGS. 1, 2, or in the form of a horizontally arranged tube, as shown in FIG. 3, or a spiral shaped tube, the coils of which are located in a horizontal plane or close to the horizontal 15 plane, as shown in FIG. 5.

When processing the finished refined alcohol with the use of the claimed method, the unit schematically shown in FIG. 4 operates as follows.

From reservoir 4 for the initial product, the water-alcohol mixture of the rectified alcohol is supplied by pump 6 into the head portion of horizontally arranged rectifying column 1. Using heating element 2, the water-alcohol mixture is evenly boiled along the entire length of column 1 on its bottom side, and with the help of cooling element 3, there is provided a uniform condensation of vapors and formation of the phlegma along the entire length of rectifying column 1 on its upper side. The phlegma flows down the side walls of the column, and packing 8 is constantly enriched with oncoming vapor of alcohol and water. In this case, the reflux number of the apparatus tends to infinity. At the same time, under the operation of pump 6, both phases, that is, liquid and gaseous ones, simultaneously move horizontally from the head portion of rectifying column 1 to the outlet thereof. The residual vapors of the water-alcohol mixture are condensed on the cooling element for finished product 7 and mixed with the phlegma. At the outlet of rectifying column 1, the finished refined alcohol is collected in the form of the cooled phlegma into reservoir 5 for the finished product. The efficiency of refining the rectified alcohol at this apparatus depends on the length of column 1, the feed rate of the initial product supplied by controlled pump 6 and the controlled temperature of heating element 2 of rectifying column.

### EXAMPLE

The test was conducted using as a rectified alcohol, namely, the 40% vodka of [symbol] [symbol] (President Standard) produced by the [symbol] [symbol] 3 (Zhytomyr LVP), Ukraine. To process the vodka with the use of the present inventive method, there was applied the apparatus shown in FIG. 4. For processing the vodka according to the method of the prototype, there was used the apparatus with a vertical arrangement of the rectifying column described as a prototype [Patent of Ukraine No. 104536, IPC: CCC12G 3/04, B01F 13/02, B01D 3/26. Publ.10.01.01.2014]. In both cases, there were used rectifying columns of the same geometric parameters and with the same packing. There was conducted a comparative analysis of the effect of the vodka processed by the method of the prototype and according to the present inventive method on the precipitation of a pig red blood cells. The test tube with pig blood, treated with 3.2% sodium citrate as an anticoagulant, was filled with the physiological dose of the vodka in concentration of 3.0 ppm, it was characteristic for a state of extreme intoxication for a human. The results were recorded using a microscope.

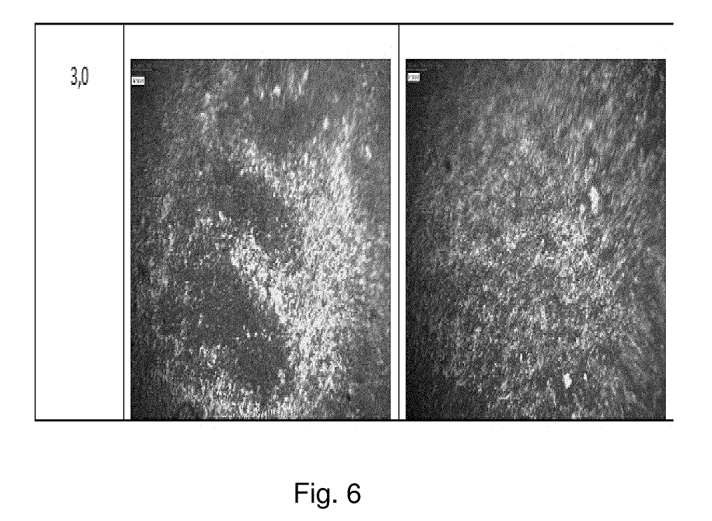

The photographs shown in FIG. 6 show that only a blood sample with rectified alcohol processed by the prototype method (picture on the left) contains a conglomerate of red blood cells.

This fact proves that processing of the rectified alcohol by the present method causes considerably lower precipitation of pig erythrocytes than when it is processed according to the prototype method.

It is known that the use of large doses of rectified alcohol by a human lead to a rapid intoxication. Physiologically, this is manifested in loss of coordination, delayed reaction, redness of the eyes, face skin, especially in the nose and cheeks, numbness of the extremities, etc. To a large extent, these symptoms are caused by the precipitation (clumping) of erythrocytes under the influence of ethanol, which leads to clogging of micro vessels and capillaries, and, accordingly, to reduced oxygen access to the brain and other vital organs. Since the body of a pig in its structure is considered to be quite close to the human body, it can be assumed that the rectified alcohol processed by the claimed method would cause less harm to the human body. This means the occurrence of a significant improvement in the quality of the alcoholic beverage. In addition, when processing the rectified alcohol by the present method, the drink does not lose the original taste of the initial product.

Based on the above, this example proves that the claimed method for refining the rectified alcohol provides maximum improvement in the quality of the drink by reducing its harmful effects on the human body while maintaining the original taste of the initial product.

Thus, the apparatus for refining the rectified alcohol with the use of the claimed method, that is, by the method of de-rectification, provides a multistage process for combining alcohol with water, wherein the de-rectified phlegma is formed in the process of heat and mass transfer between alcohol and water during their numerous phase transitions along the entire length of the rectifying column. The apparatus can be used for producing sorting of alcoholic beverages, as well as for producing or processing any alcoholic beverages or liquid medicines prepared on the basis of rectified spirit.

The proposed method and the apparatus for its implementation allow reach as follows:

The maximum improvement of the drink by reducing its harmful influence on the human body, while maintaining the original taste of the initial product;

The creation of such a design of the device for refining the rectified alcohol, which ensures the high efficiency of the processes of de-rectifying and homogenizing the drink evenly over the entire volume of the rectifying column.

It is clear that the above-mentioned embodiments of the design implementation of the inventive concept and the examples of the technological capabilities thereof don't cover all the concrete designs and all possible aspects of the industrial application of the unit in accordance with the invention, and in no event, they don't limit the rights of the inventor.

1. A method for refining rectified alcohol by passing the alcohol through a rectifying column of a device and separating a rectified alcohol condensate in the form of a phlegma, wherein the rectified alcohol is fed into a head portion of the rectifying column, with a temperature gradient being maintained inside thereof, and a finished product is collected in the form of a phlegma at an outlet of the rectifying column, which is characterized by that (wherein) the temperature gradient is created across the width of the rectifying column, and the rectified alcohol is directed horizontally along a length of the rectifying column and perpendicularly to the temperature gradient.

2. An apparatus for implementing the method according to claim 1 comprising:
a rectifying column including a packing and having end openings for feeding an initial product and discharging a finished product in the form of a phlegma; and
a heating element; and
a cooling element;
wherein the rectifying column is horizontally arranged, and the heating and cooling elements are located on the opposite sides of the rectifying column along the entire length thereof.

3. The apparatus according to claim 2, wherein the rectifying column has a form of a horizontal hollow plate, with the heating element being arranged on a lower end of the horizontal hollow plate throughout all the area thereof, and with the cooling element being arranged on an upper end of the horizontal hollow plate throughout all the area thereof.

4. The apparatus according to claim 2, wherein the rectifying column has a form of a tube twisted into a flat horizontal spiral shape with the heating element being arranged under the tube and with the cooling element being arranged above the tube.

5. The apparatus according to claim 2, wherein the rectifying column has a form of a tube spirally twisted around the vertical heating element, and the cooling element is a fan.
